(19) United States
(12) Patent Application Publication
(10) Pub. No.: US 2019/0101539 A1
(43) Pub. Date: Apr. 4, 2019

(54) METABOLOMIC BASED BIOMARKERS FOR COLON CANCER DETECTION

(71) Applicants: David CONTI, Los Angeles, CA (US); Fredrick SCHUMACHER, Cleveland, OH (US); Stan LOUIE, Fullerton, CA (US); Isaac ASANTE, Monterey Park, CA (US)

(72) Inventors: David CONTI, Los Angeles, CA (US); Fredrick SCHUMACHER, Cleveland, OH (US); Stan LOUIE, Fullerton, CA (US); Isaac ASANTE, Monterey Park, CA (US)

(73) Assignee: UNIVERSITY OF SOUTHERN CALIFORNIA, Los Angeles, CA (US)

(21) Appl. No.: 15/545,657

(22) PCT Filed: Jan. 22, 2016
(86) PCT No.: PCT/US2016/014619

§ 371 Date: Jul. 21, 2017

Related U.S. Application Data
(60) Provisional application No. 62/107,309, filed on Jan. 23, 2015.

(51) Int. Cl. G01N 33/574; G01N 33/82; G01N 33/68; G01N 30/72

(52) U.S. Cl. CPC G01N 33/57419; G01N 30/7233; G01N 33/6815; G01N 33/82

(57) ABSTRACT

A method of identifying subjects with colorectal cancer (CRC) is provided. The method includes obtaining a sample from a subject, determining a level of one or more folate one carbon metabolism (FOCM) metabolites in the sample of the subject, and comparing the level of the FOCM metabolites in the sample of the subject to a level in a non CRC control.

### RELATED APPLICATIONS

This application is a 35 USC § 371 National Stage application of International Application No. PCT/US2016/014619 filed Jan. 22, 2016; which claims the benefit under 35 USC § 119(e) to U.S. Application Ser. No. 62/107,309 filed Jan. 23, 2015. The disclosure of each of the prior applications is considered part of and is incorporated by reference in the disclosure of this application.

### STATEMENT REGARDING FEDERALLY SPONSORED RESEARCH

This invention was made with government support under Contract No. RO1 CA140561-01 awarded by National Cancer Institute. The government has certain rights in the invention.

### BACKGROUND

Colorectal cancer (CRC) is the second leading cause of cancer death in the US.¹ Most CRC patients are diagnosed late due to lack of predictive blood-based biomarkers and poor screening rate attributable to the inconvenience of current screening methods². To enhance earlier detection, there is the need for blood-based biomarkers that will facilitate early detection and further insights into the pathogenesis of CRC.

Colorectal cancer (CRC) is a cancer that evolved as a consequence of uncontrolled cell growth in the colon or rectum. These malignancies may develop as a consequence pre-existing benign adenomas where genetic alterations promote the transition from normal to cancerous growth. Epigenetic events have been recognized as an important mechanism regulating oncogene activation or silencing of tumor suppressor genes. DNA methylation is one epigenetic event that regulates expression of gene. Specifically, hypermethylation of tumor suppressor gene often impairs binding of transcription factors which regulates cell cycle and proliferation³. The DNA methylation process is highly dependent on two factors 1) methyltransferase transcription factors and 2) methyl substrates produced in the folate one carbon metabolism (FOCM)

FOCM provides the one-carbon substrate for numerous intracellular reactions critical for biosynthesis and gene regulation. Folate and its metabolites can also help maintain genomic stability through regulating DNA biosynthesis, repair and methylation. Folates are the primary methyl donors that transferring them onto substrates essential for intracellular transmethylation reactions including those involved in DNA methylation and DNA biosynthesis. S-adenosyl methionine (SAM), the primary methyl donor used for DNA methylation, is critical component found in the FOCM⁴. The region that is on the DNA dictates the type of biological response(s). In cancers, differential DNA methylation may occur at the promoters specifically along the cytosine-phosphate-guanine (CpG) islands or CpG islands shore (2 kb from CpG island). Aberrant DNA methylations may occur as a consequence of global hypomethylation, which has been associated with chromosomal instability. Alternatively, hypermethylation in targeted regions can potentially silence tumor suppressor genes thereby permitting to cellular transformation to neoplasm.

Folate and its metabolites have been described to maintain genomic stability through regulating DNA biosynthesis, repair and methylation⁵. Folate-associated one-carbon metabolism (FOCM) provides the methyl groups for numerous intracellular reactions that are critical for gene regulation through DNA methylation. Other vitamin B metabolites serve as critical co-factors in the enzymatic reactions in the FOCM.

Studies have found high dietary folates intake to be associated with decreased colorectal cancer risk⁶. There are other metabolites in the pathway that may influence the “methylation capacity” of a cell, thus influencing cancer development. These FOCM-related metabolites have been specifically quantified in an assay and further used to in a screening assay for CRC.

Current methods to quantify folates and co-factors found in the FOCM used bacterial based assays. These assays use mutant microbiological organism(s) deficient in their ability to make these specific co-factors or folates, where growth curves correlate with the level of intermediates. A major drawback is that bacterial or enzyme linked immunosorbent assay (ELISA) based assays are unable to distinguish the specific metabolites that are found in the FOCM. Liquid chromatograph mass spectrometry (LC-MS) has advanced epidemiologic studies where the components or metabolites can be quantified simultaneously are commonly referred to as metabolomics.

### SUMMARY OF THE INVENTION

One aspect of the present invention is directed to a method of identifying subjects with colorectal cancer (CRC). The method includes obtaining a sample from a subject, determining a level of one or more folate one carbon metabolism (FOCM) metabolites in the sample of the subject, and comparing the level of the FOCM metabolites in the sample of the subject to a level in a non CRC control. The comparison may be used to identify whether a the subject has CRC, and, in some embodiments, the stage of the CRC. As such, in some embodiments, the method further comprises identifying whether a subject has CRC, and optionally, the stage of the CRC based on the comparison of the level of the FOCM metabolites to a level in a non CRC control.

The sample is preferably a blood-based sample. More preferably, the sample is a plasma sample.

In one embodiment, the one or more FOCM metabolites include pyridoxine, 4-pyridoxic acid, pyridoxal, pyridoxal phosphate, 5-methyltetrahydrofolate, tetrahydrofolate, flavin mononucleotide, folic acid, dihydrofolate, riboflavin, S-adenosyl methionine, S-adenosyl homocysteine, homocysteine, cystathione and methionine.

In another embodiment, the one or more FOCM metabolites include 4-pyridoxic acid.

In another embodiment, the one or more FOCM metabolites include S-adenosyl homocysteine.

In another embodiment, the one or more FOCM metabolites include 5-methyltetrahydrofolate.

In another embodiment, the determination of the level of the one or more FOCM metabolites includes conducting a liquid chromatograph mass spectrometry (LC-MS) assay.

In another embodiment, the method includes adding a stabilization agent to the sample of the subject.

In another embodiment, the stabilization agent includes ascorbic acid and/or zinc sulfate.

In another embodiment, if the level of the one or more FOCM metabolites in the sample of the subject is statistically different than the level in the non CRC control, the subject is a candidate for CRC therapy.

In another embodiment, if the level of the one or more FOCM metabolites in the sample of the subject is at least 1.5 times greater than the level in the non CRC control, the subject is a candidate for CRC therapy.

In another embodiment, the method includes determining the level of two or more FOCM metabolites in the sample of the subject.

In another embodiment, a ratio of two FOCM metabolites in the sample of the subject is compared to a ratio of the two FOCM metabolites in the non CRC control.

Another aspect of the present invention is directed to a method of quantifying folate one carbon metabolism (FOCM) metabolites in a sample from a subject. The method includes adding a stabilization agent to the sample of the subject, determining a level of one or more folate one carbon metabolism (FOCM) metabolites in the sample of the subject by conducting a liquid chromatograph mass spectrometry (LC-MS) assay, and adjusting the determined level of the FOCM metabolites to a level at a time of collection of the sample.

In one embodiment, the one or more FOCM metabolites include pyridoxine, 4-pyridoxic acid, pyridoxal, pyridoxal phosphate, 5-methyltetrahydrofolate, tetrahydrofolate, flavin mononucleotide, folic acid, dihydrofolate, riboflavin, S-adenosyl methionine, S-adenosyl homocysteine, homocysteine, cystathione and methionine.

In another embodiment, the one or more FOCM metabolites include 4-pyridoxic acid.

In another embodiment, the one or more FOCM metabolites include S-adenosyl homocysteine.

In another embodiment, the stabilization agent includes ascorbic acid and/or zinc sulfate.

In another embodiment, the method includes determining the level of two or more FOCM metabolites in the sample of the subject.

In another embodiment, a ratio of two FOCM metabolites in the sample of the subject is compared to a ratio of the two FOCM metabolites in a control sample.

In another embodiment, the sample is a blood-based sample, and preferably a plasma sample.

In another embodiment, the method further includes comparing the adjusted level of the FOCM metabolites in the sample of the subject to a level in a control sample.

Other aspects and advantages of the invention will be apparent from the following description and the appended claims.

### BRIEF DESCRIPTION OF THE FIGURES

FIG. 1 shows the LC-MS chromatogram of tetrahydrofolate using a multiple analyte assay or metabolomics approach.

FIG. 2 shows the LC-MS chromatogram of folic acid using a multiple analyte assay or metabolomics approach.

FIG. 3 shows the LC-MS chromatogram of 5-methyltetrahydrofolate using a multiple analyte assay or metabolomics approach.

FIG. 4 shows the LC-MS chromatogram of dihydrofolate using a multiple analyte assay or metabolomics approach.

FIG. 5 shows the LC-MS chromatogram of methotrexate using a multiple analyte assay or metabolomics approach.

FIG. 6 shows the LC-MS chromatogram of pyridoxamine phosphate using a multiple analyte assay or metabolomics approach.

FIG. 7 shows the LC-MS chromatogram of pyridoxal using a multiple analyte assay or metabolomics approach.

FIG. 8 shows the LC-MS chromatogram of pyridoxamine using a multiple analyte assay or metabolomics approach.

FIG. 9 shows the LC-MS chromatogram of pyridoxine using a multiple analyte assay or metabolomics approach.

FIG. 10 shows the LC-MS chromatogram of 4-pyridoxic acid using a multiple analyte assay or metabolomics approach.

FIG. 11 shows the LC-MS chromatogram of pyridoxal phosphate using a multiple analyte assay or metabolomics approach.

FIG. 12 shows the LC-MS chromatogram of riboflavin using a multiple analyte assay or metabolomics approach.

FIG. 13 shows the LC-MS chromatogram of flavin mononucleotide using a multiple analyte assay or metabolomics approach.

FIG. 14 shows the LC-MS chromatogram of cyanocobalamin using a multiple analyte assay or metabolomics approach.

FIG. 15 shows the difference between healthy subjects and CRC patients.

FIG. 16 shows healthy subjects (circular dots) versus CRC patients using PC1 and PC2 comparison.

### DESCRIPTION

A “biomarker” as used herein refers to a molecular indicator that is associated with a particular pathological or physiological state. The “biomarker” as used herein is a molecular indicator for cancer, more specifically an indicator for colorectal cancer (CRC).

As used herein the term “cancer” refers to or describes the physiological condition in mammals that is typically characterized by abnormal and uncontrolled cell division or cell growth. Examples of cancer include but are not limited to, carcinoma, lymphoma, blastoma, sarcoma, and leukemia. More specific examples of such cancers include breast, brain, bladder, prostate, colon, intestinal, squamous cell, lung, stomach, pancreatic, cervical, ovarian, liver, skin, colorectal, endometrial, salivary gland, kidney, thyroid, various types of head and neck cancer, and the like.

As used herein, a “subject” is preferably a human, non-human primate, cow, horse, pig, sheep, goat, dog, cat, or rodent. In all embodiments, human subjects are preferred. The “subject” may be at risk of developing CRC, may be suspected of having CRC, or may have CRC. In addition, a “subject” may simply be a person who wants to be screened for CRC.

Using metabolomics approaches, plasma samples from cases and controls in the candidate gene pathway-based study were analyzed for FOCM metabolites. The quantified levels were explored for any association between FOCM metabolites and CRC risk. Biomarkers were developed that can be used to diagnose or predict CRC development. Previously, microbiological assays were used to quantify FOCM intermediates such as folates and B vitamins as pooled substrates, instead of individual metabolites. An objective of the present invention was to develop a potential biomarker predictive of CRC development and probe the pathogenesis of CRC.

It is believed that specific metabolites of B2, B12 and folates, which are components found in the FOCM, are altered when transitioning from normal epithelial cells to adenomatous polyp with terminal transformation into CRC.

In order to probe this controversy, a more sensitive and specific validated assay like LC-MS is required. The present invention provides an LC-MS-based metabolomics assay that quantifies the plasma levels of the relevant FOCM intermediates that may affect the methylation capacity thereby picking up any compensatory mechanisms that may arise due to an imbalance. The plasma levels of synthetic folic acid, 5-methyl tetrahydrofolate (SMTHF), homocysteine (Hcy), S-adenosyl methionine (SAM) and S-adenosyl homocysteine (SAH) are relevant components of FOCM that may drive the DNA methylation process. The SMTHF is the secondary methyl donor for the DNA methylation processes. The SAM/SAH ratio measures the methylation capacity of the cell with the Hcy levels associated with intracellular toxicity. Synthetic folic acid may accumulate and yield an inhibition on the folate receptors⁷, especially under reduced DHFR activity thereby affecting the levels of intracellular folates required to drive the cytosolic FOCM. Unmetabolized folates in plasma may also be associated with reduce natural killer cell cytotoxicity⁸.

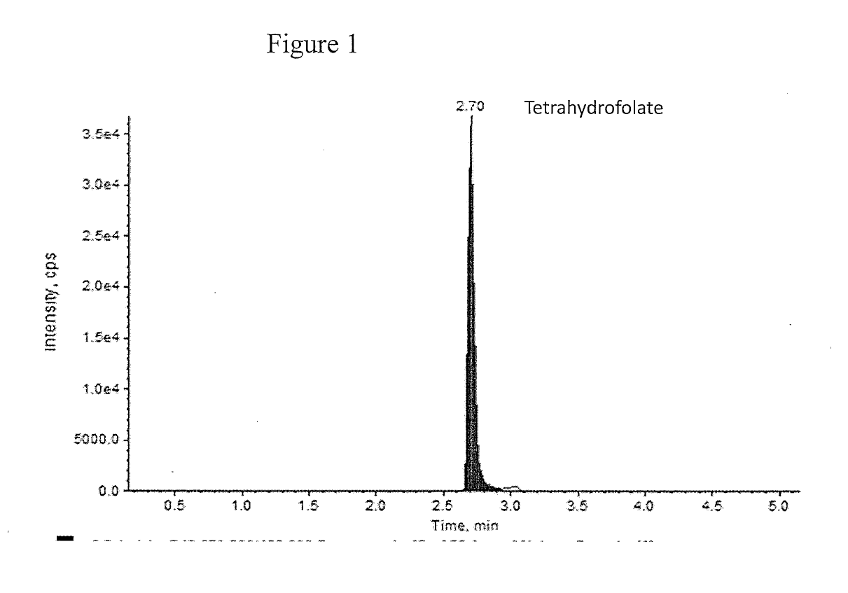

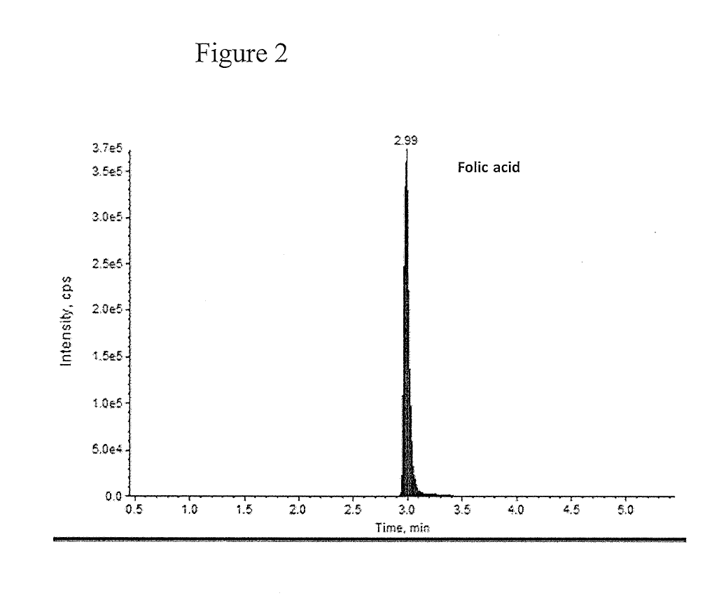

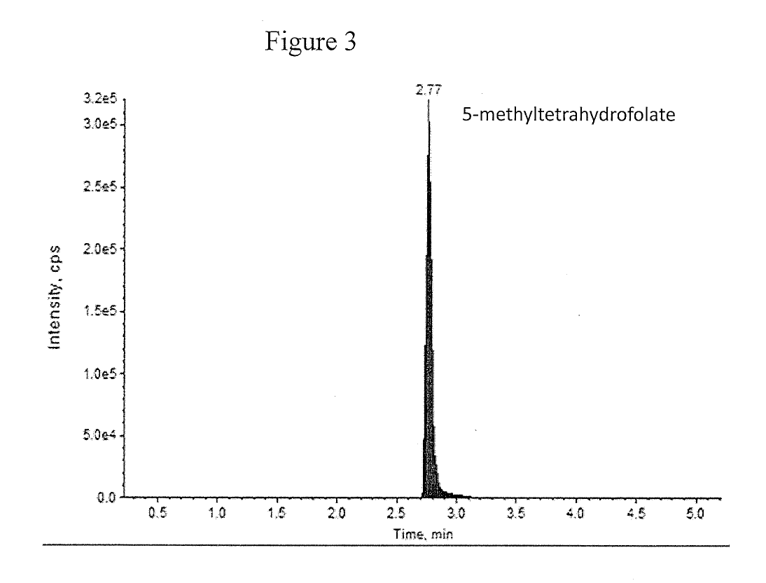

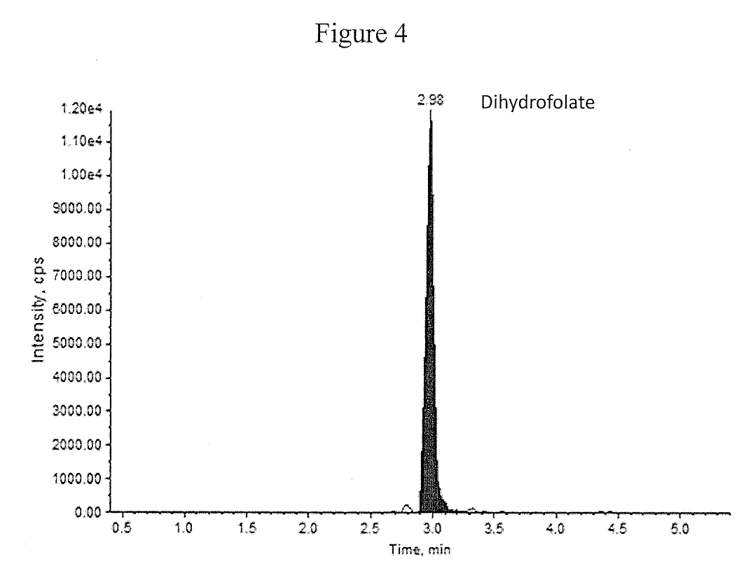

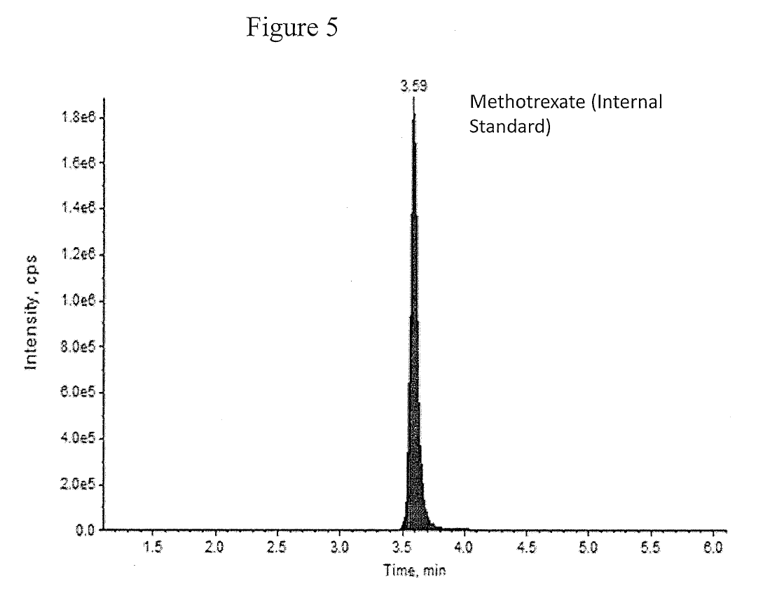

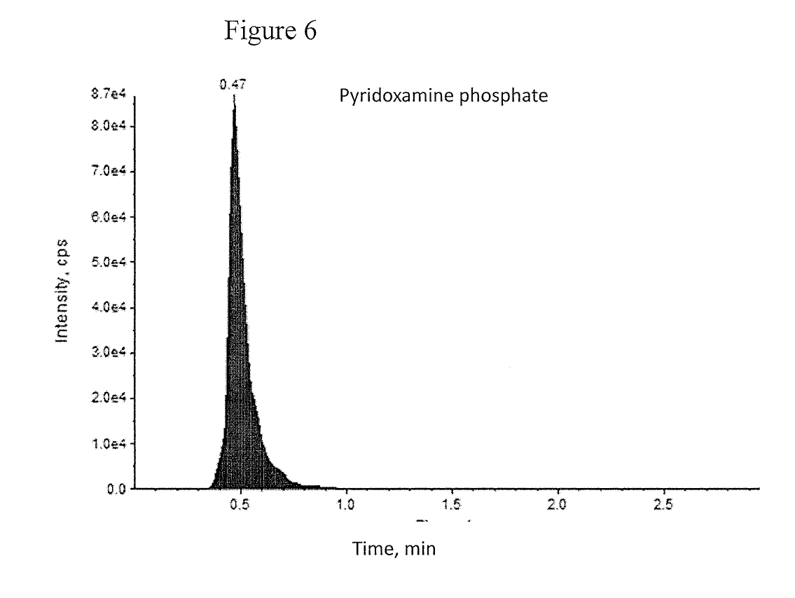

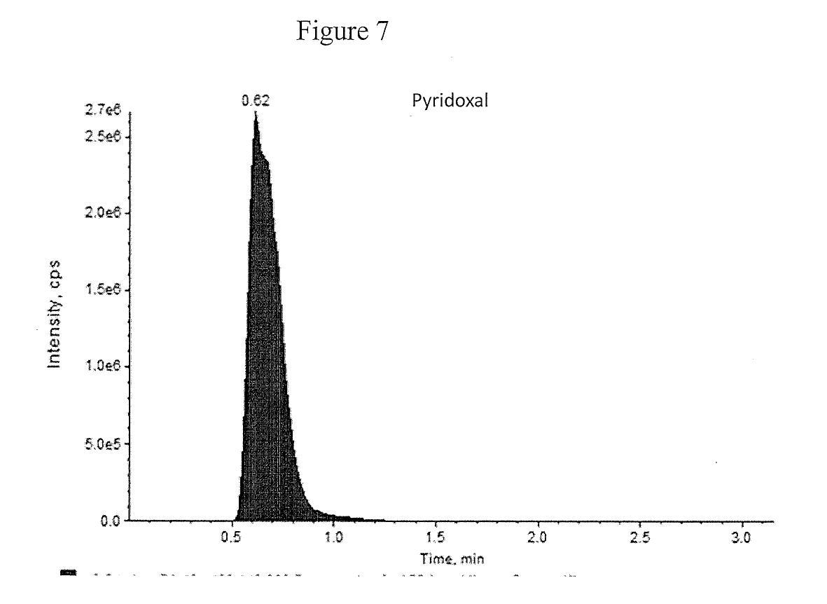

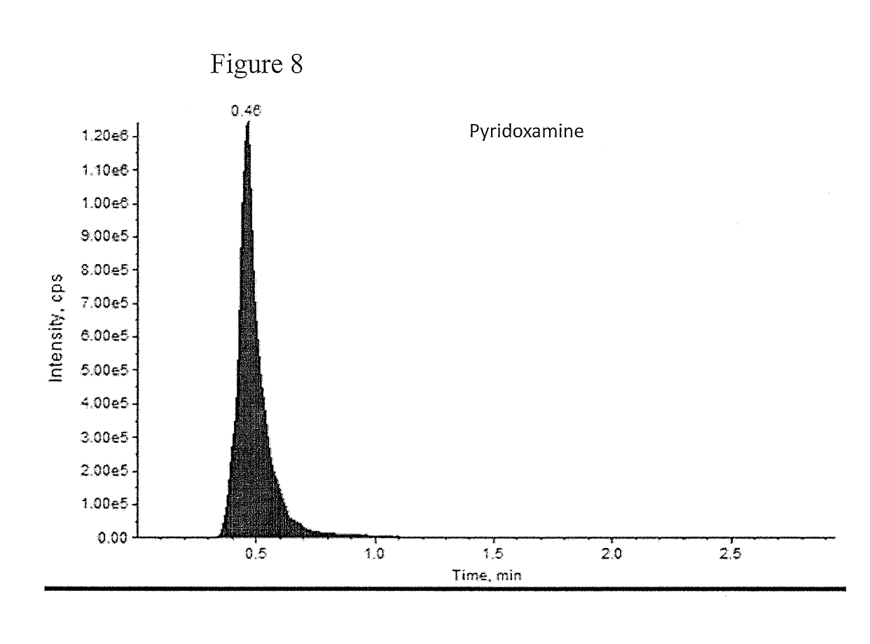

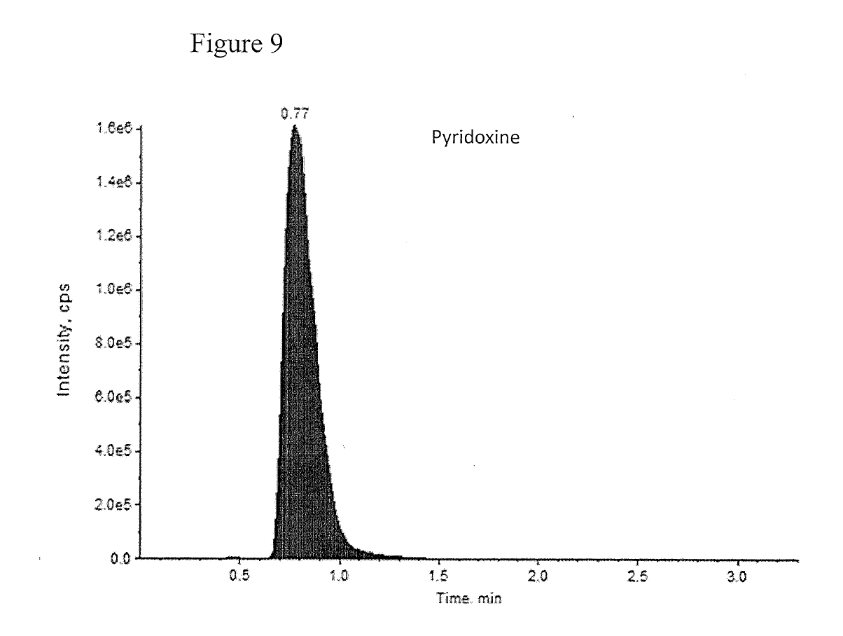

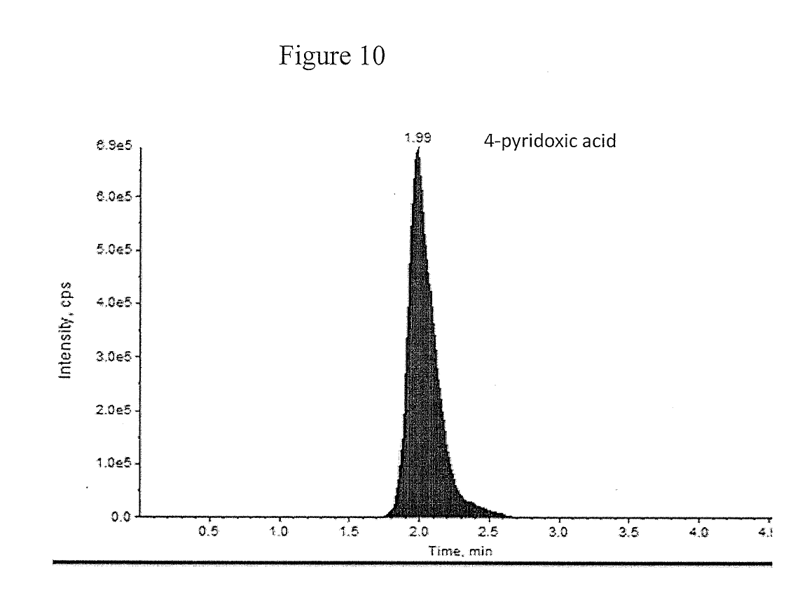

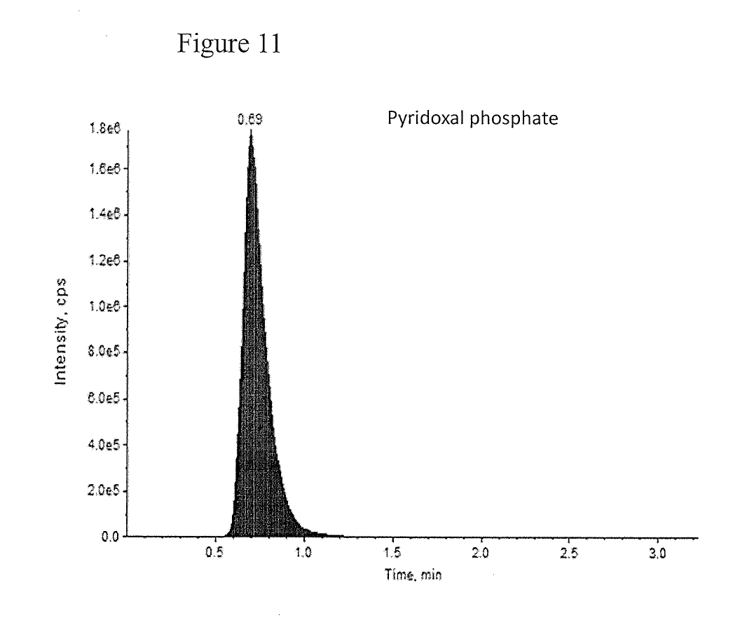

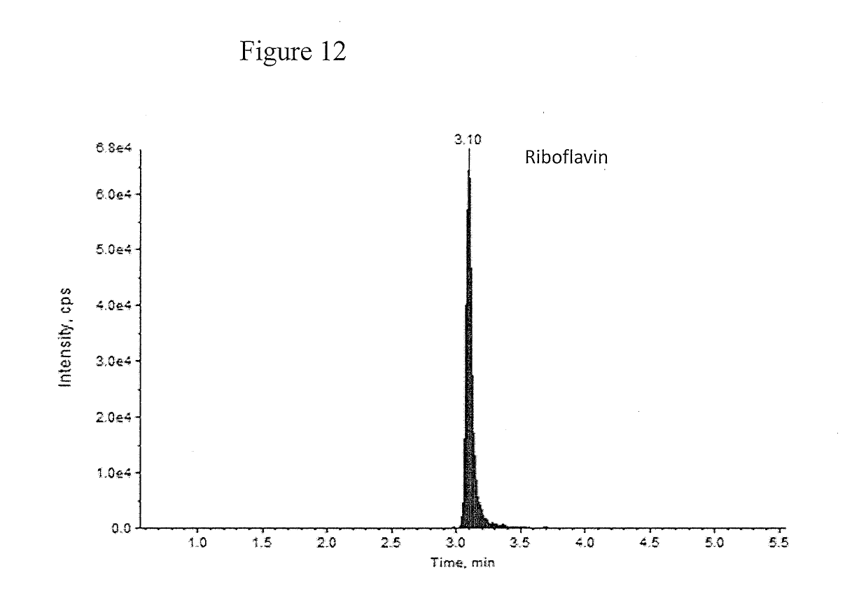

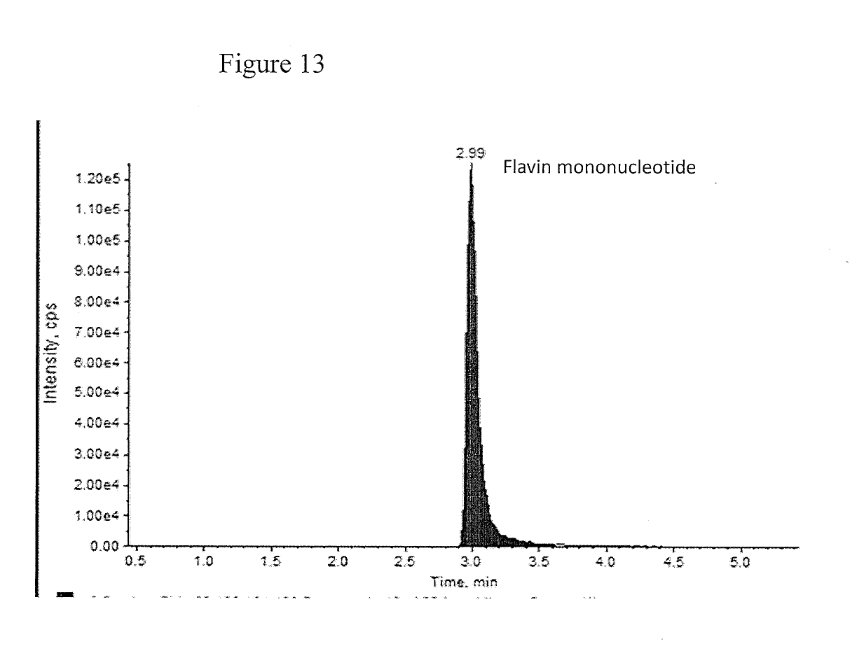

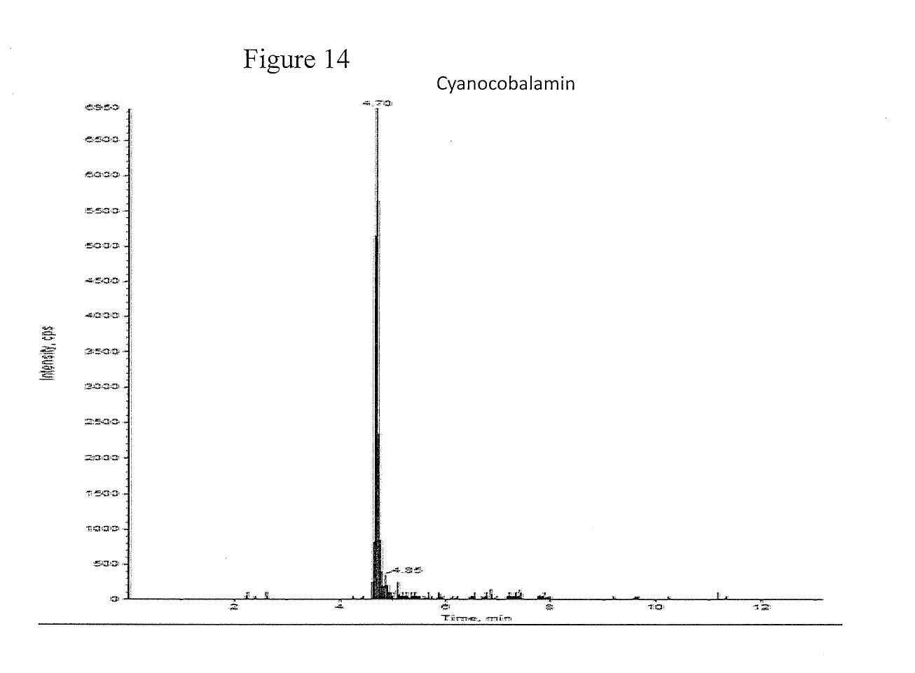

A validated metabolomics-based LC-MS assay has been developed to effectively quantify and explore the plasma levels of FOCM intermediates in CRC cases and controls who participated in the candidate gene pathway-based study. In order to make the assay economically useful in clinical setting, the intermediates were combined into cost-effective composite assays. The chromatograms shown in FIGS. 1-14 depict an assay that quantifies 13 different metabolites in a single run (methotrexate being the internal standard).

The selectivity of the assay for the individual folates facilitates the measure of each for them for association with the incidence of CRC. Vitamins B2, B6 and B12 serve as critical cofactors in the FOCM, the absence of which relevant enzymes involved in the one-carbon metabolism are impaired. The metabolites of these vitamins role as the active forms for cofactor activity and offer good estimation of the equilibrated levels of the parent compound for metabolic processes. Their quantification is necessary to explain the corresponding effect of intermediates on the methylation capacity of cells. The calibration curve for each intermediate obtained from a single run is shown in Table 1.

TABLE 1
Validation Curve for the Folate, Vitamin B2 and B6 LC-MS Assay

| | | | | | Concentration (ng/mL) | | | |
|---|---|---|---|---|---|---|---|---|
| | | Calibration Equation | | | | | Ref | Ref¹ |
| | Analyte | Gradient | Intercept | R² | LLQ | ULQ | LL | UL |
| | Cyanocobalamin | 0.0080 | 0.003 | 0.995 | 0.016 | 16 | 0.16 | 0.95 |
| Flavin | Flavin mononucleotide | 0.0003 | 0.002 | 0.998 | 0.230 | 230 | 1.32 | 5 |
| | Riboflavin | 0.0088 | 0.124 | 0.998 | 0.230 | 230 | 1.02 | 19 |
| Folate | Folic Acid | 0.0027 | 0.003 | 0.997 | 0.054 | 54 | 3.00* | 16* |
| Metabolites | Dihydrofolate | 0.0010 | 0.000 | 0.999 | 0.054 | 54 | 3.00* | 16* |
| | 5-Methyltetrahydrofolate | 0.0057 | 0.033 | 0.998 | 0.054 | 54 | 3.00* | 16* |
| | Tetrahydrofolate | 0.0005 | 0.006 | 0.991 | 0.054 | 54 | 3.00* | 16* |
| Metabolites of | Pyridoxine | 0.0137 | −0.033 | 0.997 | 0.300 | 300 | 5.00 | 30 |
| Vitamin B6 | Pyridoxal | 0.0040 | 0.021 | 0.999 | 0.300 | 300 | 5.00 | 30 |
| | Pyridoxal-Phosphate | 0.0005 | 0.009 | 0.999 | 0.300 | 300 | 5.00 | 30 |
| | Pyridoxamine | 0.0147 | −0.068 | 0.994 | 0.300 | 300 | 5.00 | 30 |
| | Pyridoxamine-Phosphate | 0.0004 | 0.003 | 0.999 | 0.300 | 300 | 5.00 | 30 |
| | 4-Pyridoxic acid | 0.0254 | 0.072 | 0.999 | 0.300 | 300 | 5.00 | 30 |

*total folates.
¹Adapted from Iverson, Christiansen, Flanagin et al, 2007; Hustard, Ueland & Soneece. 1999.

Cyanocobalamin serves as a cofactor for the transfer of a methyl group from 5-MTHF to homocysteine (Hcy) via the B12-dependent enzyme methionine synthase (MTR) and its partner methionine synthase reductase (MTRR). As adenosylcobalamin, B12 is used for the isomerization of methylmalonyl Co-A to succinyl Co-A in a reaction catalyzed by methylmalonyl Co-A mutase. The vitamin B12-specific metabolite, methyl malonic acid (MMA) is specific to this pathway with high plasma levels correlating with vitamin B12-deficiency and would be quantified as part of the relevant metabolites. The plasma levels of MMA will reliably facilitate the investigation of the role of B12-dependent enzymes like MTR in FOCM. Methylmalonic acid (MMA) can be determined using the modified assay method described by Hempen, Wanschers. This method will be validated and used to detect underivatized MMA extracted from plasma using protein precipitation. The m/z for MMA and deuterated MMA were detected at 117.1→73.0 and 120.1→76.0 respectively monitoring in the negative electron spray ion (ESI) mode. The validation parameters will be established for the calibration curve over 16× the concentration range and utilized in the plasma analysis.

A major challenge with the FOCM intermediates during storage and analysis is their poor stability. The metabolites may be unstable through environment exposures such as heat, light and/or oxygen, thereby posing great challenge to determine the actual concentration of these metabolites at the time of collection. These FOCM intermediates make the metabolomics-based assay a very powerful tool to a more accurate quantification. The instability of these vitamins pose a great challenge due to their poor stability in the presence of metallic ions, oxidative species and light which catalyze most of the degradation reactions. Most of the vitamin B and folate metabolites degrade due as a consequence of photooxidation reactions which becomes a challenge for assay development necessitating the provision of the most suitable conditions for storage and processing of the plasma samples in order to produce a highly sensitive and reliable assay method. In order to address the instability issues during sample processing, the analysis will be conducted on at 4° C. using black eppendorf tubes. A number of stabilizing agents have been evaluated where 0.5% ascorbic acid was found to be more effective in stabilizing the FOCM metabolites from oxidation, while minimizing chemical interactions with the analytes. Consistent metabolites extraction is achieved using protein precipitation approach followed by the supernatant evaporated to dryness under nitrogen. The Prominence ultra-flow liquid chromatography system used for sample analysis has an inbuilt degasser system to exclude air from the metabolites in addition to a refrigerated autosampler unit.

Since the LCMS method yields a highly selective assay, it is critical to assess the degradation kinetics of the analytes over the period of storage to be able to determine the levels of these metabolites during the collection period. This will further enhance the clinical prediction ability. The stability of these plasma vitamin B and folate metabolites were studied to validate the reliability of the assay and the time-dependent effect on the concentration of the analytes in patient samples. Using freshly made samples and assessing their degradation over time, we have established an Arrhenius models that will allow the team to extrapolate the metabolite level over time.

The stability of these metabolites were assessed using accelerated stress conditions which involves determining degradation rates of analytes through monitoring changes in time of their concentration in solution at several predetermined storage temperatures and then analyzing the results in terms of the Arrhenius equation:

k=Ae^{-E/RT}

where:
k is the specific reaction rate constant
A is the Arrhenius pre-exponential (frequency) factor
E is the activation energy [kJ/mole] or [kcal/mole]
R is the universal gas constant
T is the absolute temperature [K].

For a valid Arrhenius model to be developed, a linear plot determining the degradation rate of analyte at given storage temperatures against reciprocals of these temperatures is needed. The pre-exponential factor A, which may be obtained by extrapolating the straight line to zero value of the temperature reciprocal, appears to be affected by factors such as exposure to pollution⁷ and light⁸, relative humidity during the ageing, and the analytes. The activation energy, directly proportional to the slope of the straight line, represents a measure of sensitivity of the degradation rate of the studied property to temperature changes. The degradation rate of the analytes at ambient conditions can be estimated by extrapolating the Arrhenius plot for the analyte concentration to storage temperature. Combined with a kinetic equation describing changes of the concentration with time, this rate may then be used to estimate the life-expectancy of the analytes.

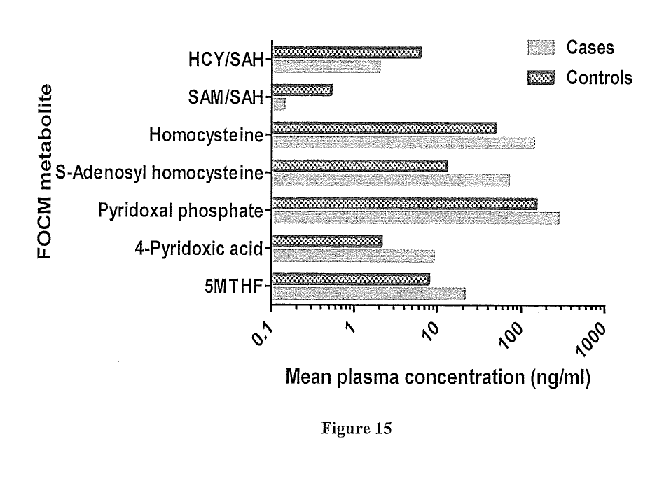

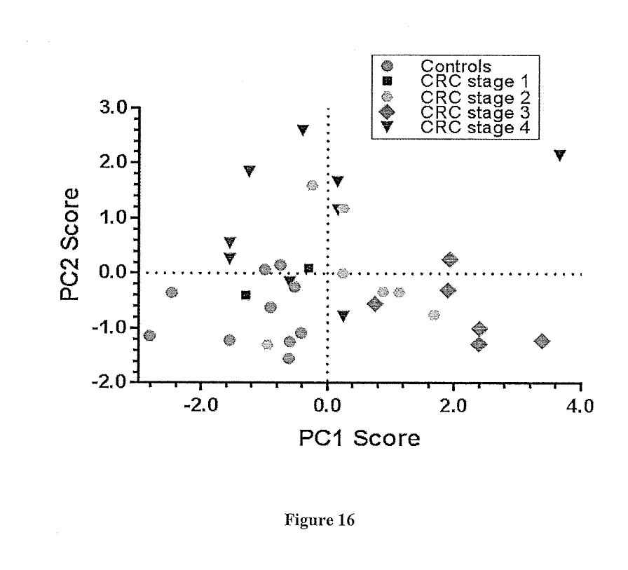

Plasma from subjects without cancer were compared with confirmed CRC patients. Their levels of FOCM metabolites were evaluated, where the levels were levels are summarized in Table 2 and FIG. 15. We have also used a PC2 Score comparing healthy subjects with CRC, where health controls have a PC2 score that <0.2, while the PC1 Score <0.0. In contrast, the majority of CRC subjects had PC1 Score >0.0 with a PC2 score >0.2 (FIG. 16).

TABLE 2
Difference between CRC Patients and Healthy Subjects

| | | Mean Plasma | | | |
|---|---|---|---|---|---|
| | | concentration (ng/ml) | | | Cohen d |
| | FOCM metabolite | Cases | Controls | p-value | Effect size^{§} |
| Folates | Folic acid | 0.33 | 0.18 | 0.26 | |
| | 5MTHF | 20.67 | 7.90 | 0.03** | 0.90 |
| | Tetrahydrofolate | 5.06 | 1.78 | 0.14 | |
| | Dihydrofolate | 96.05 | 53.99 | 0.05 | |
| | Total Folates | 107.48 | 51.66 | 0.01** | 0.72 |
| B6 metabolites | Pyridoxine | 1.56 | 0.07 | 0.46 | |
| | 4-Pyridoxic acid | 8.89 | 2.12 | 0.01** | 1.03 |
| | Pyridoxal | 71.47 | 27.06 | 0.09 | |
| | Pyridoxal phosphate | 274.40 | 151.47 | 0.01** | 0.80 |
| | Total vit B₆ | 355.29 | 180.69 | 0.005** | 0.93 |
| Flavins | Riboflavin | 6.42 | 4.10 | 0.13 | |
| | Flavin mononucleotide | 30.14 | 15.20 | 0.08 | |
| | Total flavins | 31.42 | 16.47 | 0.09 | |
| Others | S-Adenosyl methionine | 6.12 | 2.92 | 0.11 | |
| | S-Adenosyl homocysteine | 69.94 | 12.81 | <0.0001** | 2.16 |
| | Cystathionine | 13.17 | 13.01 | 0.98 | |
| | Homocysteine | 140.08 | 48.58 | 0.01** | 1.67 |
| | Methionine | 564.48 | 378.10 | 0.16 | |
| Ratio of | SAM/SAH | 0.14 | 0.52 | 0.01** | 0.80 |
| metabolites | DHF/THF | 26.07 | 33.16 | 0.66 | |
| | MET/HCY | 2.22 | 0.24 | 0.42 | |
| | 5MTHF/THF | 5.25 | 4.44 | 0.78 | |
| | HCY/SAH | 1.96 | 6.15 | 0.03** | 0.12 |
| | HCY/CYS | 7.18 | 11.46 | 0.51 | |

** Analytes whose mean values are significantly different in cases and controls. Significance was determined with a p-value <0.05.
^{§}This is a measure of clinical relevance of the analytes that show statistical significance. The level of relevance may be termed small (effect size < 0.3), medium (0.3 < effect size < 0.7) and large (effect size > 0.8)

We have developed a liquid chromatograph mass spectrometry (LC-MS) based multi-analyte assay that is able to determine the endogenous vitamins and their metabolites found in Table 2. This assay is able to quantify the levels of each metabolite. This is accomplished through stabilizing the vitamins and their metabolites using chemical stabilizers (0.2 M Zinc Sulfate) and ascorbic acid. This assay is able to distinguish the difference between healthy subjects and CRC patients with different stages of colorectal cancers. There is currently no LC-MS based assay that is able to differentiate between colorectal cancers and normal healthy subjects. In the patients we have studied where the results are summarized in Table 2, we have found that circulating concentrations of pyridoxine and its metabolites are elevated when compared to healthy subjects. Additionally, folate metabolites and flavin mononucleotide were significantly different between the two groups.

An important aspect of the present invention is that the metabolites in the LC-MS have been stabilized to allow the data to be relevant.

Another important aspect of the present invention is that samples collected in the past can be extrapolated back to the original levels using the Arrhenius equation.

Another important aspect of the present invention is that the metabolomics assay that was developed is able to differentiate between healthy subjects and those with colorectal cancers.

### Additional Experimental Details

### Patient Samples

This approach involves the metabolite profiling and comparative analysis of plasma samples from CRC and healthy controls. Plasma samples were bought from a vendor and frozen till analysis.

### Sample Preparation

The levels of FOCM components used 100 μL of plasma sample, to which 50 μL of 30 ng/mL methotrexate (internal standard) was added, and the entire sample was protein precipitated with 80% Methanol with 0.2M Zinc Sulphate. Exactly 450 μL of supernatant was evaporated to dryness under nitrogen and reconstituted in 30 μL 1% ascorbic acid.

LCMS Assay for Folate, B₆ and B₂ Metabolites

An aliquot of 20 μL aliquot was injected into an HPLC (Prominence, Shimadzu) coupled to a triple-quadruple tandem mass spectrometer (Sciex API 4000, Applied Biosystems) equipped with an electrospray ionization interface. The LC-MS/MS analysis was performed using a Sciex API 4000 triple quadrupole MS/MS system (Applied Biosystems) operating in electrospray ionization (ESI) mode coupled to Prominence UFLC system (Shimadzu) with temperature controlled autosampler. The separation of sample components was carried out using Phenomenex Kinetex 2.6 μm XB C-18 (75 mm×3.0 mm) column with an attached guard column packed with the same stationary phase (Thermo Scientific, USA). The mobile phases were as follows: A, 0.1% (v/v) formic acid; and B, acetonitrile. The flow rate was 0.2 mL/min. The gradient started at 100% mobile phase A, decreased to 90% within 1 min, when the flow rate was increased to 0.3 mL/min. The mobile phase B was increased gradually to 20% by the 14 min. The column was then flushed with 100% B for 6 min and regenerated with 100% A for an additional 10 min. The total analysis time was 33 min. The sample temperature in the autosampler was maintained at 4° C., and the injection volume was 10 μL in each run. The detection and quantification of the metabolites were performed with a positive electrospray ionization technique using the selected MRM mode. Methotrexate was used as internal standards for the assay. The MultiQuant software (AB Sciex) was used for quantification and calculations.

### Materials and Methods of Calibration

### Chemicals

The metabolite standards cyanocobalamin, riboflavin, flavin mononucleotide, folic acid, dihydrofolate, tetrahydrofolate, 5-methyltetrahydrofolate, pyridoxine, pyridoxal, pyridoxal phosphate, pyridoxamine, pyridoxamine phosphate and 4-pyridoxic acid and all other common chemicals were purchased from Sigma (St Louis, Mo., USA) and Cayman Chemicals (Ann Arbor, Mich., USA). Methotrexate, purchased from Enzo Life Sciences (Farmingdale, N.Y., USA), was used as an internal standard for the assay.

### Concentration Adjustment of Standards

To optimize the accuracy of the estimated concentrations from the calibration curves of the assay, the standards were tested for cross-contamination of other analytes. Folic acid standard was found to contain 7% of dihydrofolate (DHF) and 7% of tetrahydrofolate (THF). The DHF standard comprised of 17% as THF whilst 9% of the pyridoxamine standard was pyridoxine.

### Preparation of Stock Solutions

A stock solution of each metabolite standard was prepared in DMSO at concentrations ranging from 0.34 to 9.6 mg/mL. All stock solutions were stored at −80° C. Solutions were combined and diluted with the appropriate stabilizing solution to give an appropriate mixture of standards for use and inhibit oxidation.

Stabilizing reagents tested included ascorbic acid, tris(2-chloroethyl) phosphate, sodium citrate and dithiothreitol.

### Standards and Quality Control Samples

A mixture of standards further termed as ‘working mixture A’ composed of 160 ng cyanocobalamin, 600 ng riboflavin, 600 ng flavin mononucleotide, 108 ng folic acid, 540 ng dihydrofolate, 108 ng tetrahydrofolate, 108 ng 5-methyltetrahydrofolate, 600 ng pyridoxine, 600 ng pyridoxal, 600 ng pyridoxal phosphate, 600 ng pyridoxamine, 600 ng pyridoxamine phosphate and 600 ng 4-pyridoxic acid per milliliter of solution. Working solution A was further used to prepare calibration and QC samples. Working solutions were prepared on the day of the experiment by diluting the freshly prepared stock solution with 1% ascorbic acid in water as diluent to form calibration standards which are factor multiples of working solution A.

Calibration standards at nominal concentration factors of 0.5, 0.25, 0.1, 0.05, 0.025, 0.01, 0.005, 0.0025, 0.001, 0.0005, 0.00025 and 0.0001 of the ‘working mixture A’ as defined above. Each standard contained 75 ng/ml of Methotrexate as internal standard. Plasma standards were prepared by spiking 50 μL of the appropriate working solution into 50 μL of pooled blank plasma. Separate working solutions were used to prepare 3 concentrations of QC samples by adding appropriate amounts of working solution each time during calibration curve and sample processing. The nominal concentration factors of the QC samples were 0.1, 0.01, and 0.001 dilutions of the working solution.

### Calibration and Equations

After the run, quantitation and analysis of peaks was done using Multiquant and MarkerView softwares (AB Sciex Bioscience) respectively. Plasma calibration standards (n=8 for analytes except Cyanocobalamin where n=6) were used to generate standard curves on 4 separate occasions. A calibration curve is a plot of the analyte peak area to internal standard peak area, as y-axis and the standard concentrations as the x-axis. The analyte to internal standard area ratios were fitted by means of linear regression with a weighting factor of 1/×2 for riboflavin, FMN and DHF but no weighting for the other analytes. The fit was considered acceptable if the mean calculated values of the calibration standards over the 4 batches for each value were 15% of the nominal values or 20% at the lower limit of quantification. The calibration standards were categorized as outliers and excluded from the standard curve if the calculated accuracies were 55% or 145% of the nominal concentration (about 3 standard deviations for 15% CV). At least 6 points were used to generate each standard curve. The gradient, intercept and R-squared for the calibration curves for each analyte in indicated in Table 1.

To quantify a metabolite for a sample, the ratio of the analyte peak area-to-area of internal standard peak is extrapolated on the calibration curve to get the plasma concentration of the sample.

### Arrhenius Equation of a Metabolite

In order to estimate the degradation rate constant, k, at the storage temperature of the plasma samples (which is usually −80° C.), a degradation experiment was conducted for FOCM metabolites at ambient temperatures of 37° C., 25° C., 4° C. and −20° C. The degradation rate experiment at −80° C. did not yield consistent results according to the pattern of the ambient temperature so the degradation constant was extrapolated from the Arrhenius model. All degradation curves were assumed to be first order. All experimental samples were stored and quantified at days 0, 3, 7, 14, 21, 28 and 42 using the developed LCMS assay. The results below (for 4-pyridoxic acid) are an example of the data obtained for each of the FOCM metabolites.

| Temp | Degradation | Temp | | 1/T | Calculated | Calculated |
|---|---|---|---|---|---|---|
| (° C.) | constant, K | (K) | Ln (K) | (K − 1) | Ln (K) | K |
| 37 | 0.021 | 310 | −3.841 | 0.0032 | −3.810 | 0.022 |
| 25 | 0.018 | 298 | −4.019 | 0.0034 | −3.977 | 0.019 |
| 4 | 0.016 | 277 | −4.164 | 0.0036 | −4.302 | 0.014 |
| −20 | 0.008 | 253 | −4.806 | 0.0040 | −4.741 | 0.009 |
| −80 | ? | 193 | ? | 0.0052 | −6.313 | 0.002 |

Value of R = 8.314 Jmol⁻¹K⁻¹
Plotting Ln(K) = Ln (A) − E/R(1/T)

It can be deduced that Ln(A)=0.3183

A=1.37

However, E=−slope*R=1279.9*8.314=10.6KJmol⁻¹

Since we have these values for 4-pyridoxic acid, it means that we can find the degradation constant, k, for every storage condition. Knowing the value of K means that one can extrapolate the concentration of the analyte back in time to the time of sampling, even if there were changes in storage temperature conditions.

### PC1 and PC2

Principal Component Analysis (PCA) is a statistical approach used to explore data by transforming the number of variables in a dataset into fewer orthogonal variables in which the original variables are highly correlated together. In this analysis, the MarkerView software (AB Sciex Bioscience) was used to import the liquid chromatography mass spectrometer (LCMS) quantitation for further analysis using the log and auto scale settings. The procedures conducted on the metabolites (as original variables) are summarized below.

The plasma concentrations of the metabolites are standardized into a uniform scale across board. An example of such standardization is to subtract the mean from each value and decide the resulting value by the mean. The covariance matrix for each data point is calculated—this is a measure of how the two variables move together. By the way,

$$cor ( X , Y ) = \frac{cov ( X , Y )}{{σ}_{X} , {σ}_{Y}} .$$

Using the covariates, the eigenvalues are then deduced from the formula

[Covariance matrix]·[Eigenvector]=[eigenvalue]·[Eigenvector]

In order to re-orient our data onto the new axes (principal components), the original data is multiplied by the eigenvector. The two major principal components (PC) that explain the highest variability in the data are plotted as the major axes (PC1 versus PC2)

### Staging of Colon Cancers

Colon cancers are staged using the TNM or tumor node and metastasis classification. These are anatomical or physical scoring system where the T represents the extend of tumor invasion into colon. In contrast, N is the number of lymph node(s) where the colon cancer is detected. The M represent the metastatic status of the colon cancer. Using these three characteristics of the cancer found in the patient, a clinical staging for the colon cancer can be obtained.

T is scored from 1 to 4, where the number represented the extend of tumor found in the bowel. This is summarized in below
T1 is where the tumor is found only in the inner layer of the bowel
T2 is where the tumor has invaded into the muscle layer of the bowel wall
T3 is where the tumor has invaded into the outer lining of the bowel wall
T4 is where the tumor has invaded through the outer lining of the bowel wall.

There are 3 stages describing whether cancer cells are detected in the lymph nodes.
N0 is where there are no lymph nodes were detected to contain cancer cells
N1 is where 1 to 3 lymph nodes close to the bowel were detect to have cancer cells
N2 is where there are cancer cells found in 4 or more nearby lymph nodes

There are 2 stages of metastases where the colon cancer has either metastasize or not
M0 is where the cancer has not spread to other organs
M1 is where the cancer has spread to other parts of the body

The staging is a combination of these anatomic characteristics of the primary colon cancer.

Stage 0 is also referred carcinoma in situ (CIS).

Stage 1 Bowel cancer is the TNM staging, this is the same as T1, N0, M0, or T2, N0, M0.

Stage 2 Bowel cancer which is sub-classified as 2a and 2b. Stage 2a tumor that has into the outer lining of the bowel wall but has no lymph node involvement or metastases. In contrast Stage 2b the tumor has penetrated through the outer lining of the outer bowel wall. Similar to Stage 2a where no nodal and metastatic involvement is found.

Stage 3 is divided in three stages, where nodal involvement is the key difference between Stage 2 and 3. No signs of metastasis is note in Stage 3 staging.

Stage 4 is colon cancer that has spread.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

### REFERENCES

The following references are each relied upon and incorporated herein in their entirety.
1. U.S. Cancer Statistics Working Group (2015). United States Cancer Statistics: 1999-2012 Incidence and Mortality Web-based Report. Atlanta, Ga.
2. Rim S H, Joseph D A, Steele C B, Thompson T D, Seeff L C. Colorectal cancer screening—United States, 2002, 2004, 2006, and 2008. MMWR Surveill Summ. 2011 Jan. 14; 60 Suppl: 42-6.
3. Bird A. DNA methylation patterns and epigenetic memory. Genes & development. 2002; 16(1):6-21.
4. de Vogel S, Schneede J, Ueland P M, Vollset S E, Meyer K, Fredriksen A, et al. Biomarkers related to one-carbon metabolism as potential risk factors for distal colorectal adenomas. Cancer Epidemiology Biomarkers & Prevention. 2011; 20(8):1726-35.
5. Levine, A. J., Figueiredo, J. C., Lee, W., Conti, D. V., Kennedy, K., Duggan, D. J., . . . & Haile, R. W. (2010). A candidate gene study of folate-associated one carbon metabolism genes and colorectal cancer risk. Cancer Epidemiology Biomarkers & Prevention, 19(7), 1812-1821.
6. Kim Y I. Folate and carcinogenesis: evidence, mechanisms, and implications. J Nutr Biochem. 1999 February; 10(2):66-88.
7. Rosenberg I H. Science-based micronutrient fortification: which nutrients, how much, and how to know? Am J Clin Nutr. 2005 August; 82(2):279-80.
8. Troen A M, Mitchell B, Sorensen B, Wener M H, Johnston A, Wood B, et al. Unmetabolized folic acid in plasma is associated with reduced natural killer cell cytotoxicity among postmenopausal women. J Nutr. 2006 January; 136(1):189-94.

What is claimed is:

1. A method of identifying subjects with colorectal cancer (CRC) comprising:
obtaining a sample from a subject;
determining a level of one or more folate one carbon metabolism (FOCM) metabolites in the sample of the subject; and
comparing the level of the FOCM metabolites in the sample of the subject to a level in a non CRC control.

2. The method of claim 1, wherein the one or more FOCM metabolites are selected from the group consisting of pyridoxine, 4-pyridoxic acid, pyridoxal, pyridoxal phosphate, 5-methyltetrahydrofolate, tetrahydrofolate, flavin mononucleotide, folic acid, dihydrofolate, riboflavin, S-adenosyl methionine, S-adenosyl homocysteine, homocysteine, cystathione and methionine.

3. The method of claim 2, wherein the one or more FOCM metabolites comprise 4-pyridoxic acid.

4. The method of claim 2, wherein the one or more FOCM metabolites comprise S-adenosyl homocysteine.

5. The method of claim 2, wherein the one or more FOCM metabolites comprise 5-methyltetrahydrofolate.

6. The method of claim 1, wherein the determination of the level of the one or more FOCM metabolites comprises conducting a liquid chromatograph mass spectrometry (LC-MS) assay.

7. The method of claim 1, wherein the method comprises adding a stabilization agent to the sample of the subject.

8. The method of claim 7, wherein the stabilization agent comprises ascorbic acid and/or zinc sulfate.

9. The method of claim 1, wherein if the level of the one or more FOCM metabolites in the sample of the subject is at statistically different than the level in the non CRC control, the subject is a candidate for CRC therapy.

10. The method of claim 1, wherein if the level of the one or more FOCM metabolites in the sample of the subject is at least 1.5 times greater than the level in the non CRC control, the subject is a candidate for CRC therapy.

11. The method of claim 1, wherein the method comprises determining the level of two or more FOCM metabolites in the sample of the subject.

12. The method of claim 11, wherein a ratio of two FOCM metabolites in the sample of the subject is compared to a ratio of the two FOCM metabolites in the non CRC control.

13. A method of quantifying folate one carbon metabolism (FOCM) metabolites in a sample from a subject comprising:
adding a stabilization agent to the sample of the subject;
determining a level of one or more folate one carbon metabolism (FOCM) metabolites in the sample of the subject by conducting a liquid chromatograph mass spectrometry (LC-MS) assay; and
adjusting the determined level of the FOCM metabolites to a level at a time of collection of the sample.

14. The method of claim 13, wherein the one or more FOCM metabolites are selected from the group consisting of pyridoxine, 4-pyridoxic acid, pyridoxal, pyridoxal phosphate, 5-methyltetrahydrofolate, tetrahydrofolate, flavin mononucleotide, folic acid, dihydrofolate, riboflavin, S-adenosyl methionine, S-adenosyl homocysteine, homocysteine, cystathione and methionine.

15. The method of claim 14, wherein the one or more FOCM metabolites comprise 4-pyridoxic acid.

16. The method of claim 14, wherein the one or more FOCM metabolites comprise S-adenosyl homocysteine.

17. The method of claim 13, wherein the stabilization agent comprises ascorbic acid and/or zinc sulfate.

18. The method of claim 13, wherein the method comprises determining the level of two or more FOCM metabolites in the sample of the subject.

19. The method of claim 18, wherein a ratio of two FOCM metabolites in the sample of the subject is compared to a ratio of the two FOCM metabolites in a control sample.

20. The method of claim 13, wherein the sample is a plasma sample.

21. The method of claim 13, wherein the method further comprises comparing the adjusted level of the FOCM metabolites in the sample of the subject to a level in a control sample.
